(19) United States
(12) Patent Application Publication
(10) Pub. No.: US 2020/0408758 A1
(43) Pub. Date: Dec. 31, 2020

(54) COMPOSITIONS AND METHODS FOR DETECTING MYCOBACTERIUM TUBERCULOSIS

(71) Applicant: THE JOHNS HOPKINS UNIVERSITY, Baltimore, MD (US)

(72) Inventors: Pankaj Kumar, Baltimore, MD (US); Gyanu Lamichhane, Towson, MD (US)

(21) Appl. No.: 16/979,209

(22) PCT Filed: Mar. 11, 2019
(86) PCT No.: PCT/US2019/021585

§ 371 Date: Sep. 9, 2020

Related U.S. Application Data
(60) Provisional application No. 62/640,750, filed on Mar. 9, 2018.

(51) Int. Cl. G01N 33/569; G01N 33/549; G01N 33/52

(52) U.S. Cl. CPC G01N 33/5695; G01N 33/549; G01N 2415/00; G01N 2333/986; G01N 33/52

(57) ABSTRACT

The present invention relates to the field of Mycobacterium tuberculosis. More specifically, the present invention provides compositions and methods for detecting M. tuberculosis. In a specific embodiment, a method comprises the steps of (a) contacting a patient sample with a solid support coated with antibodies to carbapenem resistance factor A (CrfA); washing unbound molecules from the solid support using a buffer; and incubating the solid support with a beta-lactamase substrate. In certain embodiments, the beta-lactamase substrate is chromogenic. In another embodiment, the method further comprises the step of visually detecting a color change from the hydrolysis of the substrate by CrfA protein bound to the antibodies on the solid support. In yet another embodiment, the method further comprises the step of measuring color intensity of the strip at 490 nm using a spectrophotometer.

### CROSS-REFERENCE TO RELATED APPLICATIONS

This application claims the benefit of U.S. Provisional Application No. 62/640,750, filed Mar. 9, 2018, which is incorporated herein by reference in its entirety.

### FIELD OF THE INVENTION

The present invention relates to the field of Mycobacterium tuberculosis. More specifically, the present invention provides compositions and methods for detecting M. tuberculosis.

### BACKGROUND OF THE INVENTION

Tuberculosis (TB) is one of the deadliest diseases worldwide and is a global concern for both developed and developing countries. According to World Health Organization (WHO) 2016 reports (Global Tuberculosis Report 2016. In: World Health Organization. 2016), TB ranks alongside HIV as a leading cause of death worldwide and 6 developing countries accounted for the 60% of new TB cases: India, China, Nigeria, Pakistan, South Africa and Indonesia. The biggest burden of TB in developing countries is due to geographical and financial barrier. Diagnostic and clinical laboratories are often poorly resourced in rural areas of developing countries (Parsons et al., 24(2) CLIN. MICROBIOL. REV. 314-50 (2011)). Patients are often obliged to make multiple visits to poorly resourced medical health care centers over a period of time to achieve the final diagnosis of TB. There is a need to develop new diagnostics methods and tools that do not need laboratory facilities or specialist training and that could be used at the point of care (POC) to allow faster diagnosis of TB and faster initiation of treatment in demographically remote areas (McNerney R. & Daley P., 9(3) NAT. REV. MICROBIOL. 204-13 (2011)).

In low and middle-income countries, sputum smear microscopy is the most widely used method for diagnosing pulmonary TB (Desikan P., 137(3) INDIAN J. MED. RES. 442-44 (2013); Jassal M. & Bishai W., 50 Suppl. 3 CLIN. INFECT. DIS. S156-64 (2010)). This method is very simple and inexpensive and identifies the most infectious patients. However, the method requires serial sputum specimens requiring patients to visit the healthcare center repeatedly to hand over sputum samples and collect results. Moreover, the sensitivity of smear microscopy is compromised in low bacterial burden sputum samples (less than 10000 CFU per ml) (Desikan et al. (2013)). This method is also not effective in detecting extra-pulmonary tuberculosis, pediatric tuberculosis and in patients co-infected with HIV and tuberculosis (Jassal et al. (2010)). Smear microscopy and culture test also does not differentiate between TB and nontubercular mycobacteria (NTMs) (Maiga et al., 7(5) PLOS ONE e36902 (2012)). In urban areas of middle-income countries, TB diagnosis by smear microscopy is usually confirmed by culture, followed by identification of Mycobacterium tuberculosis strain and drug susceptibility testing. However, culture testing requires a resourceful laboratory with experienced staff, adequate infrastructure and biosafety measures that are completely lacking in resource-poor clinics and medical centers in remote areas of middle and low-income countries. One highly advanced point of care Nucleic Acid Amplification test (NAA), Loop mediated Isothermal Amplification (LAMP), has the potential to be accessible and cost effective for diagnosis of TB (Iwamoto et al., 41(6) J. CLIN. MICROBIOL. 2616-22 (2003)). However, LAMP tests require parallel culture and drug sensitivity testing to monitor progress of disease. Moreover, performance of NAATs including LAMP has not been found acceptable on smear negative samples and non-viable M. tuberculosis. There are several other advanced method for diagnosis for TB that are difficult to take to resource-less countries (Parsons et al. (2011)). There is a need to develop new diagnostic methods that (a) replace sputum based smear microscopy; (b) Rapid point of care test; (c) simple and easy to use by health workers; and (d) work with battery or no electricity, that can be best suitable to use in poorly-resourced countries.

### SUMMARY OF THE INVENTION

Timely detection of tuberculosis (TB) could save millions of lives in developing countries. Lack of resources in clinics and health centers in demographically remote areas of developing and poor countries is one of the main reasons for high mortality rates in TB. TB diagnostics tools that are used in resource-less health centers are not highly effective in detection of Mycobacterium tuberculosis, the bacteria that causes TB. There is a need to invent new diagnostic technologies that are very simple, highly selective and sensitive forts tuberculosis, easy to be taken to resource-less clinics and are not costly. In our current research, we have developed a molecular method towards development of point of care diagnosis of TB. The method detects a very unique marker from M. tuberculosis, CrfA (Carbapenem Resistance Factor A), which is absent in non-tubercular mycobacteria (NTMs). In particular embodiments, the detection of CrfA in M. tuberculosis culture is measured by colorimetric assay based on the hydrolysis of a chromogenic substrate by the enzyme. The molecular method is highly specific for the detection of M. tuberculosis up to as low as 10² CFU in a 25 μl sample. The technology can be utilized as a point of care diagnostic tool for detection of TB from sputum and body fluids.

For development of new TB diagnostic tools, we searched for a novel molecular marker that only exist in M. tuberculosis and that could be detected by colorimetric assays. We discovered a new class of enzyme from M. tuberculosis, CrfA (carbapenem resistance factor A), which is highly conserved in M. tuberculosis, M. bovis and M. bovis BCG (Kumar et al., 61(3) ANTIMICROB. AGENTS CHEMOTHER. E02234-16 (2017)). CrfA enzyme shows a nitrocefin hydrolysis activity that can be monitored by change in color of nitrocefin substrate to red. As described herein, we have developed a molecular method to detect M. tuberculosis by detecting the presence of CrfA enzyme in the cell lysate through monitoring its nitrocefin hydrolysis activity. The method is highly specific in the detection of M. tuberculosis, but not NTMs. This technology has a great scope in the development of diagnostic kit for rapid point of care detection of TB in remote health care centers in developing countries.

Accordingly, in one aspect, the present invention provides kits for detecting CrfA. The kits can be used to detect M. tuberculosis in a patient or subject. In one embodiment, a kit comprises (a) a solid support coated with antibodies to carbapenem resistance factor A (CrfA); and (b) a beta-lactamase substrate. In another embodiment, the kit further comprises instructions for using the kit to detect CrfA in a sample obtained from a patient. In certain embodiments, the solid support can be a strip, a disk or a multi-well plate. In a specific embodiment, the solid support is a strip. In particular embodiments, the kit further comprises a buffer for washing unbound protein following a contacting step between the patient sample and the solid support. In certain embodiments, the beta-lactamase substrate is chromogenic. In a specific embodiment, the chromogenic beta-lactamase substrate is nitrocefin.

In another aspect, the present invention provides methods for detecting CrfA. Such methods can be used to diagnose patients as having M. tuberculosis or TB. In one embodiment, a method comprises the steps of (a) contacting a patient sample with a solid support coated with antibodies to CrfA; (b) washing unbound molecules from the solid support using a buffer; and (c) incubating the solid support with a beta-lactamase substrate. In certain embodiments, the beta-lactamase substrate is chromogenic. In particular embodiments, the method further comprises the step of visually detecting a color change from the hydrolysis of the substrate by CrfA protein bound to the antibodies on the solid support. In other embodiments, the method further comprises the step of measuring color intensity of the solid support at 490 nm using a spectrophotometer. Any color sensing device can be used to measure the color intensity of hydrolyzed beta-lactamase substrate. The patient sample can be blood, serum, plasma or urine. In particular embodiments, the solid support can be a strip, a disk or a multi-well plate. In a specific embodiments, the solid support is a strip. In the methods of the present invention, the beta-lactamase substrate is chromogenic. In a specific embodiment, the chromogenic beta-lactamase substrate is nitrocefin.

### BRIEF DESCRIPTION OF THE FIGURES

FIG. 1A-1B. Principle of designing of CrfA strip and testing. FIG. 1A—CrfA protein blotted on nitrocellulose membrane immobilizes anti-CrfA polyclonal antibodies. Immobilized antibodies can bind CrfA enzyme from mycobacterial cell lysate and can detected by hydrolysis of chromogenic nitrocefin substrate. FIG. 1B—Procedure of testing CrfA strip on M. tuberculosis. FIG. 1C—CrfA strip was tested on pure CrfA protein. 25 μl pure CrfA marker protein with variable concentrations was incubated with CrfA strips for 1 minute at room temperature. After washing for 60 seconds, CrfA strips were incubated with 100 μl of 1 mM nitrocefin substrate for 5 and 16 hours at room temperature. Nitrocefin hydrolysis was monitored by change in red color and was also quantified on microplate reader at 490 nm wavelength. Reactions in the tubes shown are for 16 hours. Control was only CrfA strip with nitrocefin substrate.

FIG. 2A-2B. In vitro detection of M. tuberculosis by CrfA strip. FIG. 2A—25 μl of lysed M. tuberculosis cultures from exponential growth phase (OD600=0.24) and stationary growth phase (OD600=0.65) were incubated with CrfA strip for 1, 2, 4 and 8 minutes at room temperature. After washing for 60 seconds, CrfA strips were incubated with 100 μl of 1 mM nitrocefin substrate for 4 hours at room temperature. FIG. 2B—CrfA strips were incubated with 25 μl of lysed M. tuberculosis cultures with variable CFU/ml. After washing for 60 seconds, CrfA strips were incubated with 1 mM nitrocefin for 16 hours at room temperature. In both experiments, nitrocefin hydrolysis was monitored by change in red color and was also quantified on microplate reader at 490 nm wavelength. The experiments were performed in duplicates. Control was only CrfA strip with nitrocefin substrate.

FIG. 3. CrfA strip does not detect NTMs: 25 μl of lysed cultures of M. abscesses, M. chelonae, M. avium and M. tuberculosis were incubated with CrfA strip for 1 minute at room temperature. After washing for 60 seconds, CrfA strips were incubated with 100 μl of 1 mM nitrocefin substrate for 16 hours at room temperature. Nitrocefin hydrolysis was monitored by change in red color and was also quantified on microplate reader at 490 nm wavelength. The experiments were performed in duplicates. Control was only CrfA strip with nitrocefin substrate.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

The present invention provides compositions and methods useful for detecting CrfA in a sample obtained from a patient or subject. In certain embodiments, the patient or subject is a mammal such as a non-primate (e.g., a cow, dog, pig, cat, dog, horse, etc.) or a primate (e.g., a human). In another embodiment, the subject is a non-human animal, such as a bird, reptile, and a non-human mammal. In another embodiment, the subject is a farm animal (e.g., a pig, horse, or cow), a pet (e.g., a guinea pig, dog, or cat) and/or a laboratory animal (e.g., a rat or mouse). In a preferred embodiment, the patient or subject is a human.

Examples of patients or subjects from which such a biological sample may be obtained and utilized in accordance with the kits and methods presented herein include, but are not limited to, asymptomatic subjects, subjects manifesting or exhibiting 1, 2, 3, 4 or more symptoms of TB, subjects clinically diagnosed as having TB, subjects predisposed to infections (e.g., subjects with a genetic predisposition to infections, and subjects that lead a lifestyle that predisposes them to infections or increases the likelihood of contracting an infection), subjects suspected of having TB, subjects undergoing therapy for TB, subjects with TB and at least one other condition (e.g., subjects with 2, 3, 4, 5 or more conditions including, but not limited to HIV), subjects not undergoing therapy for TB, and subjects that have not been diagnosed with TB.

A biological sample can be obtained from any tissue or organ in a patient or subject, or a secretion from a patient or subject. Representative biological samples from a subject include, without limitation, nasal swabs, throat swabs, feces, dermal swabs, blood (including blood culture), serum, plasma, sputum, saliva, bronchio-alveolar lavage, bronchial aspirates, lung tissue, spinal fluid, synovial fluid and urine. In a specific embodiment, a sample comprises a blood sample. In another embodiment, a sample comprises a plasma sample. In yet another embodiment, a serum sample is used. In a further embodiment, a urine sample is used. Techniques for collecting biological samples are known to those of skill in the art.

Moreover, a sample obtained from a patient or subject can be divided and only a portion may be used as described herein. Further, the sample, or a portion thereof, can be stored under conditions to maintain the sample for later analysis. The definition of “sample” also includes samples that have been manipulated in any way after their procurement, such as by centrifugation, filtration, precipitation, dialysis, chromatography, treatment with reagents, washed, or enriched for certain cell or protein populations. The terms further encompass a clinical sample, and also include cells in culture, cell supernatants, tissue samples, organs, and the like.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any antigen-binding fragment or derivative of any of the herein described antibodies. The antibody can be a single chain variable fragment (scFv), a dimeric scFv, a Fab, a Fab′, a F(ab′)2 fragment or a full length antibody.

As used herein, the term “antigen” is generally used in reference to any substance that is capable of reacting with an antibody. More specifically, as used herein, the term “antigen” refers to CrfA. An antigen can also refer to a synthetic peptide, polypeptide, protein or fragment of a polypeptide or protein, or other molecule which elicits an antibody response in a subject, or is recognized and bound by an antibody.

As used herein, the terms “binding agent specific for” or “binding agent that specifically binds” refers to an agent that binds to a marker, e.g., CrfA, and does not significantly bind to unrelated compounds. Examples of binding agents that can be effectively employed in the disclosed methods include, but are not limited to, lectins, proteins and antibodies, such as monoclonal or polyclonal antibodies, or antigen-binding fragments thereof, aptamers, etc. In certain embodiments, a binding agent binds a biomarker with an affinity constant of, for example, greater than or equal to about 1×10⁻⁶ M. Kits and methods of the present invention can include or utilize binding agents.

The terms “specifically binds to,” “specific for,” and related grammatical variants refer to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷M, 10⁻⁸M to 10⁻⁹ M, 10⁻¹⁰ M, 10⁻¹¹ M, or 10⁻¹² M.

The compositions and methods of the present invention involve a contacting step in which a patient sample is contacted with a solid support such as a strip, disk, or multi-well plate, that is coated with binding agents, e.g., antibodies, to CrfA. In particular embodiments, the contacting step of the methods presented herein is conducted at about 20° C. to about 42° C., about 25° C. to about 40° C., about 25 to about 35° C., and about 30° C. to about 35° C.

The present invention further involves a detection step for detecting CrfA protein bound to the antibodies coating the solid support strip, membrane, multi-well plate, and the like. Because CrfA hydrolyzes β-lactam substrates, certain embodiments involve the detection of CrfA protein using such substrates.

The detection step of the kits/methods presented herein may be performed for the minimum amount of time needed for enough substrate to be utilized by a sample positive for CrfA to allow detection by a standard method for a particular detectable substrate (e.g., visual observation and/or spectrophotometry for a chromogenic beta-lactamase substrate). In particular embodiments, a washing step can be performed between the contacting and detection steps to remove unbound protein and other components present in the sample.

In particular embodiments, the detection step is performed for or about 2 minutes to about 60 minutes, for or about 2 minutes to about 45 minutes or for or about 2 minutes to about 30 minutes, for or about 2 minutes to about 15 minutes, and more specifically for or about 2 minutes to about 10 minutes after the contacting step. In another embodiment, the detection step of the methods presented herein is performed for or about 15 minutes to about 5 hours, for or about 30 minutes to about 5 hours, for or about 30 minutes to about 4 hours, for or about 30 minutes to about 5 hours, for or about 1 hour to about 2 hours, for or about 1 hour to about 3 hours, for or about 1 hour to about 4 hours, for or about 1 hour to about 5 hours, for or about 2 hours to about 4 hours, for or about 2 hours to about 5 hours, or for or about 2 hours to about 6 hours after the contacting step. In another embodiment, the detection step of the methods presented herein is performed at different time points for or over about 24 hours, for or about 20 hours, for or about 18 hours, for or about 16 hours, for or about 12 hours, for or about 8 hours, for or about 4 hours, for or about 2 hours or for or about 1 hour.

In some embodiments, a qualitative difference between the test strip, for example, and optionally a control is compared when determining substrate utilization. For example, if a chromogenic beta-lactamase substrate, such as nitrocefin, is used in the kits/methods of the present invention, whether or not the test strip, disk, membrane, plate well, etc., turns a particular color (e.g., red for nitrocefin) may be assessed. In other embodiments, a quantitative difference between the test strip and optionally a control is compared when determining substrate utilization. For example, if nitrocefin, a chromogenic beta-lactamase substrate, is used in the compositions, the percentage of the substrate that turns red may be assessed.

The particular technique used to assess substrate utilization will vary depending upon the substrate chosen. For example, if the detectable beta-lactamase substrate is a chromogenic substrate (e.g., nitrocefin or PADAC®), then substrate utilization can be assessed by visual observation or spectrophotometry (at, e.g., a wavelength of or about 490 for nitrocefin). In particular, the hydrolysis of nitrocefin can be, for example, detected by visually observing the color of the substrate turning from yellow to red. The hydrolysis of PADAC® can be detected by visually observing the color of the substrate turning from violet to yellow. If the detectable beta-lactamase substrate is a fluorogenic substrate, then substrate utilization can be detected by measuring the fluorescence of the substrate. Different antibiotics have different wavelengths and the use of an antibiotic as a substrate by a bacterial species will result in a change in the wavelength of the composition. Thus, in some embodiments, a spectrophometer is used to monitor changes in the wavelength of a composition comprising an antibiotic as the beta-lactamase substrate.

Thus, substrates for use in the detection of CrfA can comprise a detectable beta-lactamase substrate. Any beta-lactamase substrate that is readily detectable may be used in the compositions presented herein. Non-limiting examples of detectable beta-lactamase substrates include chromogenic substrates, fluorogenic substrates, and antibiotics. Chromogenic beta-lactamase substrates include, but are not limited to, nitrocefin (3-[2,4-dinitrostyryl]-7-(2-thienylacetamido]3-cephem-4-carobxylic acid (Calbiochem, San Diego, Calif.)), PADAC® (Pyridinium-2-azo-p-dimethylaniline chromophore (Calbiochem. San Diego, Calif.)), CENTA™ (EMD Chemicals, Inc., San Diego, Calif.), HMRZ-86 ((7R)-7[2-aminothiazol-4-yl]-(z)-2-(1-do)-3-(2,4-dinitrostyryl)-3-cephem-4-carboxylic acid trifluoroacetate, E-isomer (Kanto Chemical Co., Inc. Tokyo, Japan)), and cefesone. Fluorogenic substrates include, but are not limited to, Fluorcillin Green 495/525 and Fluorocillin Green 345/350 LIVE BLAZER™-FRET B/G (Invitrogen, Carlsbad, Calif.). Antibiotics include beta-lactams, penicillin, amoxicillin, etc. In a specific embodiment, the concentration of substrate that exhibits substrate utilization as detected by a technique known to one of skill in the art, such as spectrophotometry and visual observation, is used in the embodiments presented herein.

In a specific embodiment, the detectable beta-lactamase substrate is nitrocefin. In a particular embodiment, nitrocefin is present in a composition described herein at a concentration of about 1 μM to about 1 mM, about 1 μM to about 750 μM, about 1 μM to about 500 μM, or about 1 to about 250 μM. In another embodiment, nitrocefin is present in a composition described herein at a concentration of about 20 μM to about 200 μM.

In another embodiment, the detectable beta-lactamase substrate is CENTA™. In a particular embodiment, CENTA™ is present in a composition described herein at a concentration of about 1 μM to about 1 mM, about 1 μM to about 750 μM, about 1 μM to about 500 μM, or about 1 μM to about 250 μM. In another embodiment, CENTA™ is present in a composition described herein at a concentration of about 20 μM to about 200 μM.

In another embodiment, the detectable beta-lactamase substrate is HMRZ-86. In a particular embodiment, HMRZ-86 is present in a composition described herein at a concentration of about 1 μM to about 1 mM, about 1 μM to about 750 μM, about 1 μM to about 500 μM, or about 1 μM to about 250 μM. In another embodiment, HMRZ-86 is present in a composition described herein at a concentration of about 20 μM to about 200 μM.

In one embodiment, each of the substrate compositions included in a kit presented herein contains the same detectable beta-lactamase substrate. In another embodiment, each of the substrate compositions included in a kit presented herein contain the same or a similar (generally within about 10% of each other) concentration of the same detectable beta-lactamase substrate. In some embodiments, the kits presented herein comprise concentrated solutions, e.g., 2×, 5× or 10× solutions of a detectable beta-lactamase substrate, which can be diluted and added to a composition. In some embodiments, the kits presented herein comprise a detectable beta-lactamase substrate as a frozen reagent. In other embodiments, the kits presented herein comprise a detectable beta-lactamase substrate as a dried reagent.

In some embodiments, the antibodies that specifically bind CrfA are bound to a solid support. In one embodiment, the solid support is a paper strip. In other embodiments, the solid support can also comprise the well of a plate, panel, cassette or tray, paper disk or a tube. Alternatively, the antibodies can be present in a liquid or lyophilized form and can be reconstituted or diluted and bound to the solid support prior to the contacting and detecting steps.

In a specific embodiment, the paper disk or paper strip is filter paper. Non-limiting examples of the types of filter paper that may be used include Whatman paper (VWR, Pennsylvania, U.S.A.). In another embodiment, the antibodies included in the kits are in dry wells that are hydrated before use. In some embodiments, substrate utilization is detected by visual inspection when the compositions included in the kits are in the form of a paper disk, a paper strip or a dry well.

Without further elaboration, it is believed that one skilled in the art, using the preceding description, can utilize the present invention to the fullest extent. The following examples are illustrative only, and not limiting of the remainder of the disclosure in any way whatsoever.

### Examples

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Bacterial cultures. Mycobacterium abscessus ATCC 19977, Mycobacterium tuberculosis H37Rv, Mycobacterium avium 104, M. chelonae ATCC 35752 were used as the primary strains in the study.

Purification of CrfA protein. Protein was purified as per published protocol (Iwaomoto et al., 2003).

Blotting of CrfA on nitrocellulose membrane. 420 pmoles of CrfA protein was loaded on SDS-PAGE gel. The protein from SDS-PAGE gel was transferred to nitrocellulose membrane (company) using wet western blotting system.

Immobilizing of antibodies to CrfA blotted nitrocellulose membrane strips. Nitrocellulose membrane was blocked with blocking buffer, 4% skimmed milk in TBST buffer, for 1-2 hours at room temperature. Membrane was further treated with 1:2 dilutions of anti-CrfA polyclonal antibodies for 2 hours at room temperature followed by slow washing for 2 hours with TBST buffer at room temperature. Finally, nitrocellulose membrane was dried at room temperature followed by cutting into strips and were named as CrfA strips. Dried CrfA strips were stored at room temperature.

Nitrocefin hydrolysis activity of CrfA strip. For testing the activity of CrfA strip on pure CrfA protein, CrfA protein with variable concentrations (1000-3.9 pmoles) was added onto dry CrfA strip followed by washing of strip for 120 seconds with TBST washing buffer to remove unbound protein from the strip. The strip was dipped in 1000 of 1 mM nitrocefin substrate in a 1.5 ml microcentrifuge tube and incubated at room temperature in dark. Hydrolysis of nitrocefin was observed visually with change in substrate color from yellow to red. The concentration of hydrolyzed nitrocefin was quantitated on microplate reader at 490 nm wavelength from its absorbance (A), molar extinction coefficient (ε) 20500 and path length (l) (0.5 cm) using the Beer's law A=εcl. Control experiment was also done to test the nitrocefin hydrolysis activity of CrfA protein blotted on CrfA strip alone.

Testing of CrfA strips on Mycobacterium tuberculosis and non-tubercular Mycobacteria (NTMs). For testing of CrfA strip on M. tuberculosis, culture grown in 7H9 broth was used to obtain bacterial cells in exponential (A600=0.24) and stationary growth phase (A600=0.65) and cells were harvested and lysed by silica beads similar to the protocol published earlier (Kumar et al. (2017)). 25 μl of lysed culture was loaded on CrfA strip, incubated for 1-8 minutes and strip was washed with TBST buffer for 60 seconds to remove non-specific proteins followed by incubation with nitrocefin substrate and colorimetric analysis of hydrolyzed nitrocefin. CrfA strip was also used for detection of M. tuberculosis with serial dilutions from 1.37×10⁶ CFU/ml to 1.37×10² CFU/ml. With similar protocol, CrfA strip were used for detection of nontubercular mycobacteria M. chelonae, M. abscessus and M. avium cultures grown on 7H9 media.

### Results

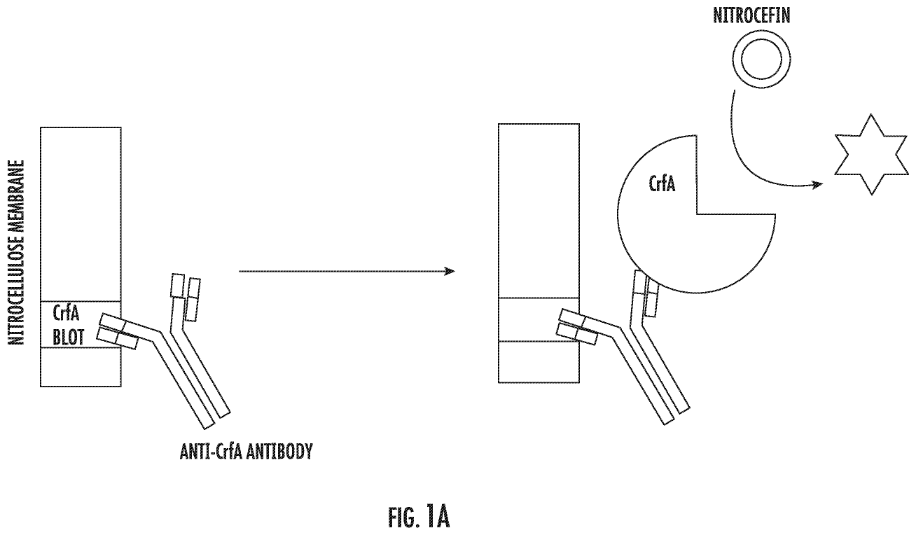

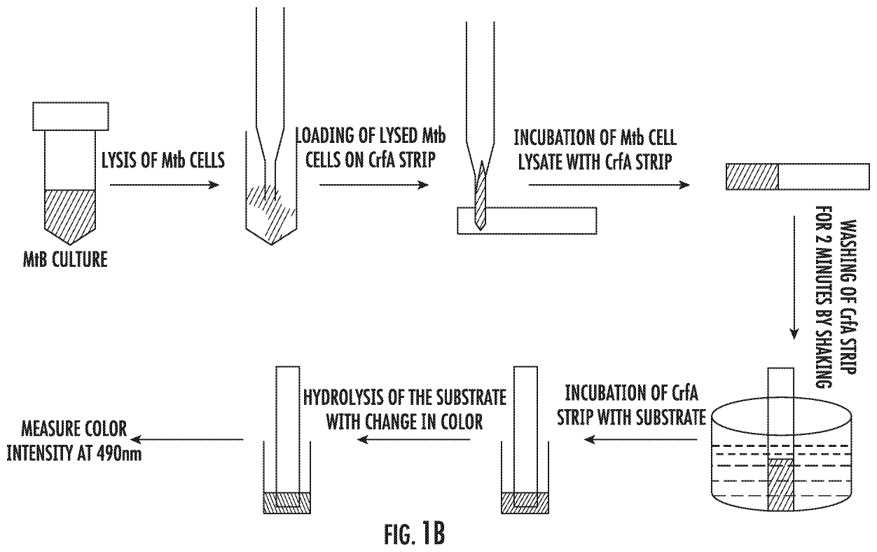

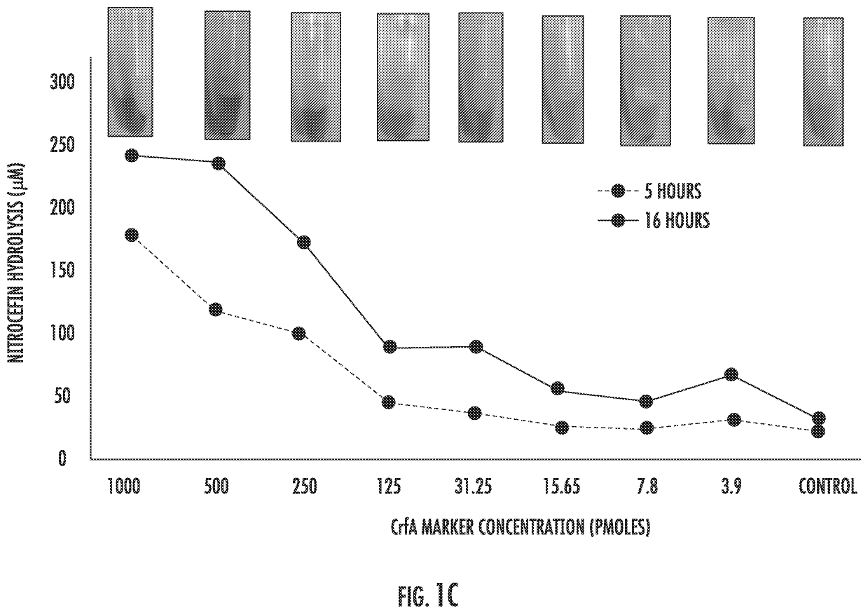

CrfA strip can detect pure CrfA protein in solution. We have developed a molecular method that has the capability to detect CrfA protein from M. tuberculosis (FIG. 1A). In the method, anti-CrfA antibodies are immobilized onto nitrocellulose membrane strips that can bind and enrich CrfA protein from M. tuberculosis whole cell lysate. Binding of CrfA protein to strips can be detected by its nitrocefin hydrolysis activity (FIG. 1A). We named these strips as CrfA strips. We have previously shown that polyclonal antibodies raised against CrfA could detect this protein both from exponential and stationary phase cultures of M. tuberculosis, but not from culture filtrate (Kumar et al. (2017)) and we presumed that CrfA is a cytosolic or cell wall protein but not a secretory protein (Kumar et al. (2017)). Therefore, CrfA strip should have the capacity to bind CrfA protein from cellular lysate of M. tuberculosis. CrfA gene is only found in M. tuberculosis and M. bovis, but not in NTMs. For the detection of M. tuberculosis, identification of unique marker is the key to success of a diagnosis. CrfA is a unique marker that can be used for detection of TB. CrfA also has the ability to hydrolyze nitrocefin substrate (Kumar et al. (2017)). CrfA strip has the ability to bind CrfA enzyme from cell lysate of M. tuberculosis that can be detected through nitrocefin hydrolysis assay, with a change in color that can be observed visually or can be quantified by spectrophotometry at 490 nm wavelength (FIGS. 1A and 1B).

We wanted to validate and quantify how much CrfA marker could be detected by CrfA strip from a solution. We tested the binding of CrfA strip on pure CrfA marker solution at room temperature and find that the marker could be detected to as low as 3.9 pmoles (FIG. 1C). We could visually detect nitrocefin hydrolysis activity for 1000-31.25 pmoles of CrfA marker in 5 hours, but could also visually detect 15-3.9 pmoles of CrfA in overnight incubation at room temperature with nitocefin substrate.

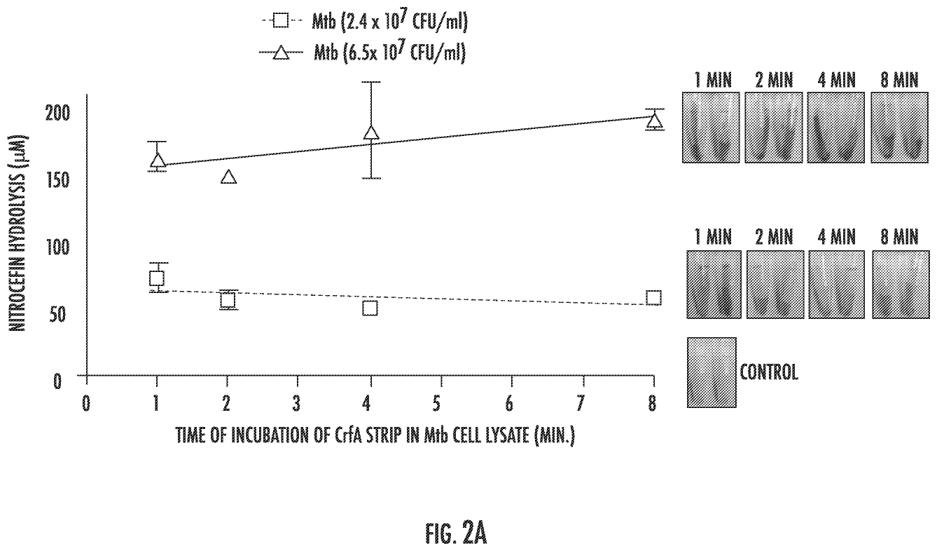

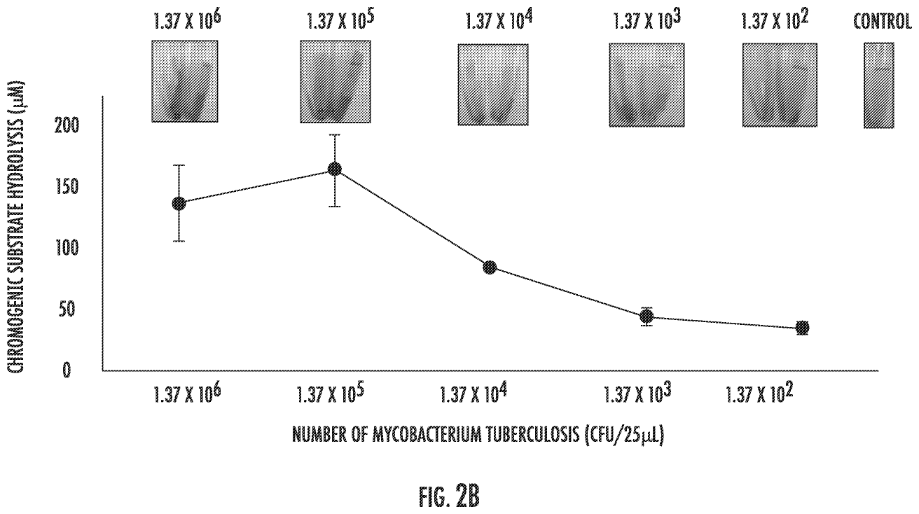

CrfA strip detects M. tuberculosis in both exponential and stationary growth phase. With CrfA strips, we tested the detection of CrfA marker from M. tuberculosis cultures. CrfA strips were incubated in bacterial culture lysates from both exponential and stationary growth phase of M. tuberculosis for 1, 2, 4 and 8 minutes at room temperature (FIG. 2A). There was ˜2-fold more detection of nitrocefin hydrolysis activity of CrfA marker with stationary growth culture. There were 6.5×10⁷ CFU/ml in stationary growth culture whereas 2.4×10⁷ CFU/ml in exponential culture and that makes a 2.7 fold difference in CFU of these two types of cultures. This difference in CFU of these two growth cultures of M. tuberculosis might be the possible explanation in fold more detection of nitrocefin hydrolysis by CrfA strip.

We further tested how many M. tuberculosis cells the CrfA strip could detect in vitro. We tested CrfA strip on 25 μl lysates of stationary growth culture of M. tuberculosis in various dilutions (1.37×10⁶ to 1.37×10² CFU/25 μl). We could visually detect the nitrocefin hydrolysis activity of CrfA strip for 1.37×10⁶-0.37×10⁴ culture dilutions in 2-4 hours (data not shown), but could also visually detect lower culture dilutions 1.3×10³ and 1.3×10² in overnight incubation with nitrocefin substrate at room temperature.

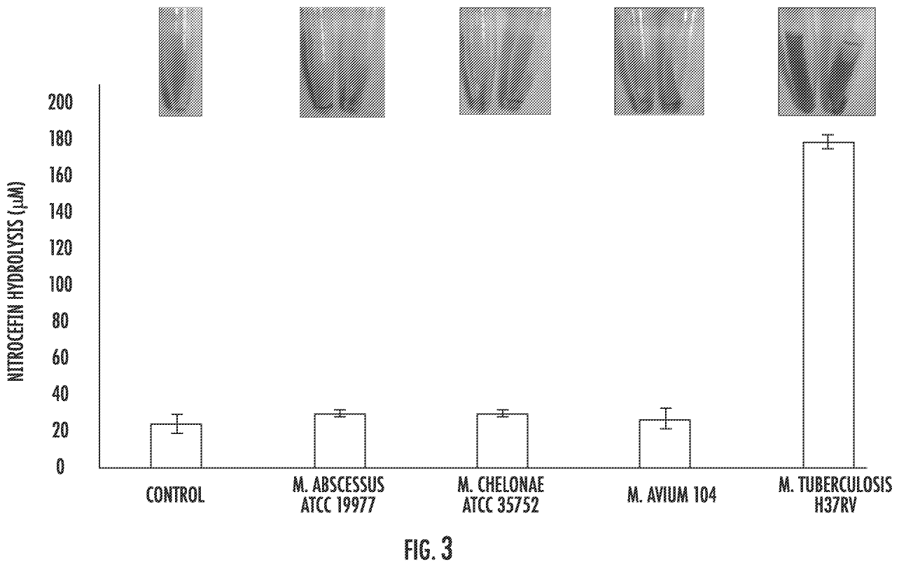

CrfA strip does not detect non-tubercular mycobacteria. It is difficult to differentiate between tubercular and non-tubercular mycobacteria with smear microscopy and culture staining method during the diagnosis of TB (Maiga et al. (2012)). We selected a few NTMs, M. abscessus, M. chelonae and M. avium for our current study. We searched for the CrfA gene in these mycobacteria and found no homology. We further tested the CrfA strips on these nontubercular mycobacteria and could not detect nitrocefin hydrolysis activity (FIG. 3). Our study clearly demonstrates that CrfA strip based molecular method only detects M. tuberculosis amongst the different mycobacterial species we used in the study.

### DISCUSSION

Recently, WHO-led consensus meetings have developed and reviewed target product profiles (TPP) for high-priority diagnostic needs that included (a) replacement of sputum based smear microscopy; (b) non-sputum based biomarker for all form of TB; (c) simple low cost triage test for use by first-contact health providers; and (d) a rapid drug susceptibility test (Denkinger et al., 211 Suppl. 2 J. INFECT. DIS. S29-38 (2015)). In our current study, we have developed a novel proof of concept (POC) based on a new molecular method for the detection of M. tuberculosis. The molecular method is based on the detection of novel marker CrfA present in M. tuberculosis, but absent in NTMs. The method also has the ability to detect M. tuberculosis cells as low as ˜10³ CFU/ml and intensity of detection can be quantified by intensity of color using spectrophotometry. Our molecular method has the ability to be developed into a rapid diagnostic tool that detect TB from sputum or body fluids, has the potential to replace sputum base smear microscopy and culture based TB test, easy to analyze and handle the detection of TB with color intensity measurement and do not show false positive with NTMs. Our molecular method for the detection of TB has the potential to develop into a diagnostic tool that can follow WHO's guidelines of TPP for high priority diagnostic needs (Denkinger et al. (2015)).

1. A kit comprising:
a. a solid support coated with antibodies to carbapenem resistance factor A (CrfA); and
b. a beta-lactamase substrate.

2. The kit of claim 1, further comprising instructions for using the kit to detect CrfA in a sample obtained from a patient.

3. The kit of claim 1, wherein the solid support is selected from the group consisting of a strip, a disk or a multi-well plate.

4. The kit of claim 1, wherein the solid support is a strip.

5. The kit of claim 1, further comprising a buffer for washing unbound protein following a contacting step between a patient sample and the solid support.

6. The kit of claim 1, wherein the beta-lactamase substrate is chromogenic.

7. The kit of claim 6, wherein the chromogenic beta-lactamase substrate is nitrocefin.

8. A method comprising the steps of:
a. contacting a patient sample with a solid support coated with antibodies to CrfA;
b. washing unbound molecules from the solid support using a buffer; and
c. incubating the solid support with a beta-lactamase substrate.

9. The method of claim 8, further comprising the step of visually detecting a color change from the hydrolysis of the substrate by CrfA protein bound to the antibodies on the solid support.

10. The method of claim 8, further comprising the step of measuring color intensity of the strip at 490 nm using a spectrophotometer.

11. The method of claim 8, wherein the patient sample is selected from the group consisting of blood, serum, plasma and urine.

12. The method of claim 8, wherein the solid support is selected from the group consisting of a strip, a disk or a multi-well plate.

13. The method of claim 8, wherein the solid support is a strip.

14. The method of claim 8, wherein the beta-lactamase substrate is chromogenic.

15. The method of claim 14, wherein the chromogenic beta-lactamase substrate is nitrocefin.
